(19) United States
(12) Patent Application Publication
(10) Pub. No.: US 2006/0147933 A1
(43) Pub. Date: Jul. 6, 2006

(54) Interspecies embryonic stem cell

(76) Inventor: Luc Schoonjans, Wilsele (BE)

Correspondence Address: CLARK & ELBING LLP, 101 FEDERAL STREET, BOSTON, MA, 02110, US

(21) Appl. No.: 10/544,169

(22) PCT Filed: Jan. 28, 2004
(86) PCT No.: PCT/BE04/00015

§ 371 Date: Jul. 28, 2005

(30) Foreign Application Priority Data
Jan. 29, 2003 (GB) 0302074.0

(51) Int. Cl. C12Q 1/68; C12N 5/06

(52) U.S. Cl. 435/6; 435/354

(57) ABSTRACT

Present invention demonstrates the derivation of ES cells from hybrid blastocysts, obtained by the mating of two different Mus species. The invention also relates to the use of these interspecies hybrid ES cells for the generation of genetically modified mammalian animals and for the identification of quantitative trait loci (QTL) associated with species specific phenotypes.

### FIELD OF THE INVENTION

The present invention relates to isolated interspecies embryonic stem cells with germ line transmission capability derivable from Mus musculus×Mus spretus hybrid mice and isolated pure groups of said cells. These embryonic stem cells (ES cells) with germ line transmission capability can be used for the generation of genetically modified intraspecies murine animals and for the identification of quantitative trait loci (QTL) associated with the species specific phenotypes. Mores specifically the invention relates to the use of these hybrid ES cells for the generation of genetically modified Mus spretus mice and for the identification of quantitative trait loci (QTL) associated with species specific (Mus musculus versus Mus spretus) phenotypes.

### BACKGROUND OF THE INVENTION

ES cell lines are cell lines derived from the inner cell mass (ICM) of blastocyst-stage embryos, which can be cultured and maintained in vitro under specific conditions for many passages, i.e. replating of cells onto new cell culture dishes at regular time intervals, without loss of their pluripotency. They maintain a normal karyotype and when reintroduced into a host blastocyst they can colonize the germline (Bradley A. Teratocarcinomas and Embryonic Stem Cells: A practical approaches (Ed. E J Robertson) JRI press Ltd., Oxford 1987, p 113-51.). Such cell lines may provide an abundance of pluripotent cells that can be transformed in vitro with DNA (see below), and selected for recombination (homologous or non-homologous) of exogenous DNA into chromosomal DNA, allowing stable incorporation of the desired gene. To date, germline transmission, i.e. the transmission of the ES genome to the next generation, has however only been achieved with ES cells of certain mouse strains.

Murine embryonic stem cells were first isolated in 1981 (Evans M J, Kaufman M H. Nature 1981; 292: 154-6 and Martin G R. Proc Natl Acad Sci USA 1981; 78: 7634-8). Since then, several ES cell lines have been established and they are now widely and successfully used for the introduction of targeted mutations or other genetic alterations into the mouse genome (Pascoe W S, et al. Biochim Biophys Acta 1992; 1114: 209-21 and Brandon E P et al. Current Biology 1995; 5: 625-34, 758-65, 873-81.). Most of the germline-competent mouse ES cell lines that are in current use have been obtained in the 129 strain, and the remainder in a few other inbred strains (C57BL/6 and crosses with C57BL/6). Furthermore, ES cell lines are at best obtained in 30% of explanted blastocysts, even in the 129 strain of mice, and success rates of around 10% appear to be closer to the norm (Robertson E J. Teratocarcinomas an Embryonic Stem Cells: A Practical Approach (Ed. E J Robertson) 1987. IRL Press, Oxford, pp 71- 112 and Nagy A, et al. Proc Natl Acad Sci USA 1993; 90: 8424-8).

The most commonly used approach to generate chimeric animals is to inject about 10-15 isolated ES cells into the blastocoel of a host blastocyst and to allow the cells to mix with the cells of the inner cell mass. The resultant chimeric blastocysts are then transferred to recipients for rearing. Alternatively diploid aggregation using very early (8-16 cell) stage embryos and tetraploid aggregation can be used as hosts for ES cells. Briefly, ES cells are ‘sandwiched’ between early stage embryos devoid of their zona pellucida, cultured overnight and implanted into a foster mother. This technique can be performed under conditions yielding either chimeric or totally ES cell-derived offspring.

Presumptive pluripotential ES cells have been isolated from a number of other species than mice, including hamster, pig, sheep, cattle, mink, rat, primate, human, chicken, marmoset, medakafish and man. In only a few instances (pig, chicken, medakafish), have these cell lines given rise to chimeras when reintroduced into blastocysts, but thus far none have given rise to germline transmission.

ES cells are maintained in an undifferentiated state by the presence of feeder layers producing various factor(s) that prevent the cells from differentiating. It has been shown that several cytokines are responsible for this effect: DIA/LIF (differentiation inhibitory activity/leukaemia inhibiting factor), interleukin-6 in combination with soluble interleukin-6 receptor, interleukin-11, oncostatin M, ciliary neurotrophic factor and cardiotrophin. It is now possible to establish and maintain ES cells in culture in the absence of feeder cells but in the presence of such factors, at least for several passages. In species other than the mouse, ES cell technology is still under development and there are no published data reporting germ line transmission in any species other than the house mouse (Mus musculus).

Presently all mouse embryonic stem cells are derived from the Mus musculus species. Date of this invention ES cells with germ line transmission capability had not been derived from other mus species

We show here for the first time the derivation of ES cells from hybrid blastocysts, said interspecies ES cells, obtainable by the mating of two different species, namely Mus musculus and Mus spretus.

### SUMMARY OF THE INVENTION

The present invention concerns isolated interspecies Mus musculus×Mus spretus hybrid embryonic stem cell (ES) with germ line transmission capability.

These interspecies Mus musculus×Mus spretus ES cells are characterised in that more than 40% of the microsatellites of its DNA are polymorphic in length, preferably more than 70% of the microsatellites of its DNA are polymorphic in length and most preferably more than 90% of the microsatellites of its DNA are polymorphic in length.

In these interspecies Mus musculus×Mus spretus ES cells the Mus spretus genomic background can be from SPRET/Ei (Spain) Ei Mus spretus strain mice and the Mus musculus genomic background can be from C57BL/6J×Mus musculus strain. The interspecies Mus musculus×Mus spretus hybrid ES cell can be from SPRET/Ei Mus spretus strain×C57BL6/J Mus musculus strain.

An embodiment of present invention is also a population of the isolated interspecies Mus musculus×Mus spretus hybrid ES cell. This can be a pure population.

In yet another embodiment of the invention the interspecies Mus musculus×Mus spretus hybrid ES cells are used for introducing mutations into the Mus spretus genome. Mutations can be specifically introduced into Mus spretus allele. The interspecies Mus musculus×Mus spretus hybrid ES cells can be used in a method for introducing mutations into the Mus spretus genome. The method can comprise a) transfection of the interspecies Mus musculus×Mus spretus hybrid embryonic stem (ES) cells with a gene-targeting construct, which specifically recombines homologously with the Mus spretus gene, b) assessing Mus musculus×Mus spretus hybrid ES cells for homologous recombination, c) generating chimeric mice by blastocyst injection d) assessing germline transmission of the Mus spretus genome and e) breeding the chimeric mice, which transmit the Mus spretus genome, to homozygosity, in a pure Mus spretus background. The introduced mutations can be null mutations, point mutations, translocations, inversions or deletions.

In yet another embodiment the interspecies Mus musculus×Mus spretus hybrid ES cells are used in a method for analysing gene function or identification of quantitative trait loci comprising the generation of radiation induced chromosomal deletion in such high polymorphism interspecies hybrid ES cells of Mus musculus×Mus spretus.

Yet another embodiment of present invention is a method for introducing mutations into the Mus spretus genome, comprising a) transfection of the interspecies Mus musculus×Mus spretus hybrid embryonic stem (ES) cells with a gene-targeting construct, which specifically recombines homologously with the Mus spretus gene, b) assessing Mus musculus×Mus spretus hybrid ES cells for homologous recombination, c) generating chimeric mice by blastocyst injection, d) assessing germline transmission of the Mus spretus genome and e) breeding the chimeric mice, which transmit the Mus spretus genome, to homozygosity, in a pure Mus spretus background. The introduced mutation can be null mutations, point mutations, translocations, inversions or deletions.

Another embodiment is a method for analysing gene function or identification of quantitative trait loci comprising the generation of radiation induced chromosomal deletion in the interspecies hybrid ES cells of Mus musculus×Mus spretus.

Yet another embodiment is an high-through put analysis system for analysing gene function for identification of quantitative trait loci, said ssyetm comprising the cell or cell population of any of interspecies hybrid Mus musculus×Mus spretus ES cells

### ILLUSTRATIVE EMBODIMENTS OF THE INVENTION

Advances in recombinant DNA technology over the last decade have greatly facilitated the isolation and manipulation of genes, to the point where any conceivable novel construct can be engineered, such as by fusing the promoter of one gene to the coding sequence of another, or by site-directed mutagenesis. Likewise, advances in embryo manipulation have facilitated the transfer of these novel exogenous genes into endogenous chromosomal DNA, generating transgenic animals. Transgenic animals can be generated either by injection of DNA into the pronucleus of zygotes, by introduction of (genetically manipulated) pluripotent embryonic stem (ES) cells into host “embryos”, and more recently by nuclear transfer with stably transfected somatic donor cells into enucleated oocytes.

Mus spretus

Presently all mouse embryonic stem cell are derived from the Mus musculus group. ES cells have not been derived from other mouse species yet.

All Mus species have the same basic karyotype of 40 acrocentric chromosomes. The three closest known relatives of Mus musculus are aboriginal species with restricted ranges within and near Europe. All three species—M. spretus, M. spicilegus, and M. macedonicus—are sympatric with M. musculus but interspecific hybrids are not produced in nature. There is a complete barrier to gene flow between the house mice and each of these aboriginal species. The ability of two animal populations to live sympatrically—with overlapping ranges—in the absence of gene flow is the clearest indication that the two populations represent different species (Nevertheless, in the forced, confined environment of a laboratory cage, Bonhomme and colleagues were able to demonstrate the production of interspecific F1 hybrids between each of these aboriginal species and M. musculus (Bonhomme F, et al. Experientia. 1978; 34: 1140-1 and Bonhomme, F, et al. Mus. Biochem-Genet. 1984; 22: 275-303.).

Mus spretus is a western Mediterranean short-tailed mouse with a range across the most southwestern portion of France, through most of Spain and Portugal, and across the North African coast above the Sahara in Morocco, Algeria, and Tunisia. M. spretus is sympatric with the Mus. musculus. domesticus group across its entire range. In 1978, Bonhomme and his colleagues reported the landmark finding that M. spretus males and laboratory strain females could be bred to produce viable offspring of both sexes. Although all male hybrids are sterile (Guenet, J L, et al. Genet-Res. 1990; 56: 163-5.), the female hybrid is fully fertile and can be backcrossed to either M. musculus or M. spretus males to obtain fully viable second generation offspring.

The species Mus spretus and Mus musculus are at an evolutionary distance of 3 million years and display great genetic polymorphisms and different stress-induced phenotypes. Therefore the embryonic stem cells of the present invention are extremely usefull in the following research fields.

Mus spretus is presently used to support research in many areas including:
Reproductive Biology Research: Fertility Defects (male progeny from outcrosses to inbred are sterile)
Research Tools: Genetics Research (Evolution and Systematics Research)
Research Tools: Genetics Research (Gene Mapping: Polymorphisms)
Research Tools: Genetic Research (Cancer and TNF resistance)

Reproductive Biology Research: Fertility Defects

In the mouse, male infertility has been found in hybrids between two species, Mus spretus and Mus musculus, from which most of the laboratory strains have been established. Understanding hybrid sterility might give an insight into not only mechanism by which a new species is evolved, but also genetic regulation of gametogenesis in males (Guenet, J L, et al. Genet-Res. 1990; 56: 163-5 and Fossella J, et al. Mamm Genome 2000; 11 (1): 8-15.).

Genetics Research (Gene Mapping: Polymorphisms)

Until a few years ago, gene mapping in the mouse was done mainly using crosses of inbred laboratory strains or RI strains. These methods were limited, however, by the low degree of polymorphism observed among laboratory strains and RI progenitors and the associated difficulty in finding a polymorphism needed for mapping. In the mid-1980s this problem was overcome through the development of interspecific crosses, which involve crosses between a laboratory strain and a distantly related species of Mus (Avner P, et al. Trends-Genet. 1988; 4(1): 18-23 and Guenet J L, et al. Curr-Top-Microbiol-Immunol. 1988; 137: 13-7.).

The high degree of genetic polymorphism present between the parents of such a cross makes it possible to map virtually any gene in a single cross. The most commonly used parents for interspecific backcrosses (IB) are C57BL/6J (the prototypic inbred strain) and Mus spretus, the most distantly related mouse species that will still form fertile hybrids with laboratory mice. The interspecific cross, involving a laboratory strain (Mus musculus) and a distantly related species Mus spretus, allowed literally thousands of genes to be mapped within the same cross.

In a series of papers, Bonhomme and colleagues have already demonstrated the power of the interspecific cross for performing multi-locus linkage analysis with molecular and biochemical markers (Bonhomme F, et al. C-R-Seances-Acad-Sci-D. 1979; 289(6): 545-8 and Bedell A M, et al Genes-Dev. 1997; 11(1): 1-10). With the large evolutionary distance that separates the two parental species, it is possible to readily find alternative DNA and biochemical alleles at nearly every locus in the genome. This finding stands in stark contrast to the high level of non-polymorphism observed at the majority of loci examined within the classical inbred lines. The significance of the interspecific cross for mouse genetics cannot be understated: it was the single most important factor in the development of a whole genome linkage map based on molecular markers during the last half of the 1980s.

Many of the major diseases affecting humans, including diabetes, cancer, epilepsy, and obesity, are not caused by single-gene mutations, but rather by the cumulative effect of mutations at several different loci. Furthermore, some of these diseases reflect a predisposition that is genetically inherited but is under significant influence from acquired somatically mutations or environmental influences. The genes that cause or predispose to such complex diseases, called quantitative trait loci (QTLs), can be dominant or recessive and act additively or epistatically to induce disease. Each QTL by itself may have only a weak effect and it is only when several QTLs are inherited by a single individual that disease or disease predisposition ensues. In general, QTL analysis requires screening of hundreds, if not thousands, of individuals and scoring them for markers scattered across the entire genome. Obviously the mouse has advantages over humans as many inbred strains are available for analysis and thousands of progeny can easily be produced by programmed breeding. (Bedell A M, et al. Genes-Dev. 1997; 11(1): 1-10.). In a typical QTL analysis of a complex trait, a mapping population is generated by crossing two highly differentiated progenitor strains or lines of mice. Most often, several hundred F2 or backcross progeny are tested and genotyped genome-wide.

A number of QTLs already have been identified in the mouse that produce phenotypes similar to human diseases, such as airway hyperresponsiveness (De Sanctis G T and Drazen J M. Am J Respir Crit Care Med 1997; 156(4Pt 2): S82-8.), alcohol and morphine preference (Berretini W H; et al. Nature Genet. 1994; 7:54-58 and Crabbe J C, et al. Science 1994, 264: 1715-1723), atherosclerosis (Hyman R W, et al. Biochem Genet 1994; 32: 397-407), epilepsy (Frankel W N, et al. Genetics 1994; 138: 481-489.), blood pressure (Zimdahl H, et al. Hypertension. 2002;39(6): 1050-2.), obesity (West D B, et al. J Clin Invest 1994; 94: 1410-1416 and Chagnon Y C, et al. Obes Res 2000; 8(1): 89-117), high density lipoprotein cholesterol levels (Wang X and Paigen B. Arterioscler Thromb Vasc Biol 2002; 22(9): 1390-1401).

Chromosomal deletions have already been shown to be powerful tools in the genetic analysis of complex genomes, enabling the systematic identification and location of functional units along defined chromosomal regions. In mice, deletion complexes created by whole animal irradiation experiments have enabled a systematic characterization of functional units along defined chromosomal regions. However, classical mutagenesis in mice is impractical for generating deletion sets on a genome-wide scale.

You et al. (You Y, et al. Methods 1997; 13(4): 409-21 and You Y, et al. Mamm Genome 1998; 9(3): 232-4) have already shown that F1 hybrid ES cells of the BALB/cTa×129/SvJae genotype and of the C57BL/6J×129/SvJae genotype retain germline colonizing ability after exposure to levels of irradiation that induce chromosomal deletions. Not only are these deletions useful for the identification of genetic functions, but they also serve as mapping reagents for existing mutations or traits.

The very high polymorphism between Mus musculus and Mus spretus make ES cell of the Mus musculus×Mus spretus genotype ideal cell lines for the generation of radiation-induced chromosomal deletions. Between different strains of Mus musculus no more than 40% of the microsatellites are polymorphic in length. Between C57BL/6 and Mus spretus up to 90% of microsatellite length can be polymorphic in length. The availability of Mus musculus×Mus spretus hybrid ES cell lines can therefore definitely contribute to a faster and more efficient high-throughput analysis of gene function and identification of quantitative trait loci.

Cancer Resistance

Different inbred mouse strains vary greatly in their susceptibility to tumour development in a variety of tissues. Intraspecific and interspecific crosses can be used to map loci that control this disposition. Crosses of Mus musculus with Mus spretus are highly resistant to tumour development in the skin, lung and lymphoid system. (Hiroki Nagaseet al. Cancer Research 61, 1305-1308, Feb. 15, 2001 and T. A. Dragani Cancer Res., Jun. 15, 2003; 63(12): 3011-3018).

Mouse models using interspecific crosses between Mus spretus, which is relatively cancer resistant, and inbred strains of Mus musculus, which are relatively cancer susceptible have been used to map cancer modifier genes (Balmain A, et al Trends in Genetics 14(4):139-144, 1998.

Mice of C57BL6/J inbred strain develop thymic lymphomas at very high frequency after acute gamma-irradiation, while mice of several inbred strains derived from the wild progenitor of the Mus spretus species and their F1 hybrids with C57BL/6J appear extremely resistant (Santos J. et al. Oncogene. 2002 Sep. 26; 21(43): 6680-3.).

Interspecific crosses between Mus musculus and Mus spretus can be used for the detection of strong genetic interactions between tumor modifier genes (. (Hiroki Nagaseet al. Cancer Research 61, 1305-1308, Feb. 15, 2001)

Tumor Necrotic Factor Resistance

The SPRET/Ei mouse strain, derived from Mus spretus, exhibits an extremely dominant resistance to TNF-induced lethal inflammation (Staelens J. et al Proc Natl Acad Sci USA. 2002 Jul. 9; 99(14): 9340-5. Epub 2002 Jun. 27.).

Thusfar, derivation of ES cells with germline transmission capability from species other than Mus musculus has not been realized yet. We report here for the very first time the derivation of ES cells with germline transmission capability from Mus musculus domesticus×Mus spretus (C57BL/6J×SPRET/Ei (Spain)) mice.

### EXAMPLES

### Example 1

### Conditioned ES Cell Culture Medium

Conditioned medium was used for derivation and culture and culture of ES cells of Mus musculus×Mus spretus hybrid mice.

The basic medium was composed of: 500 ml DMEM high glucose, 70 ml fetal bovine serum, 13 ml penicillin/streptomycin, 13 ml glutamine, 6.3 μl β-mercaptoethanol, and 13 ml non-essential amino acids. Conditioning the basic medium with Rab9 or Rab9#19 fibroblast cells.

A) ES Cell Culture Medium Preconditioned with Rab9

Basic ES cell medium, conditioned by confluent monolayer cultures of the Rab9 fibroblast cells (ATCC CRL-1414), is collected for 4 consecutive days and the conditioned media are pooled for use in ES cell culture. Each day 15 cm Petri dishes are refreshed with 25 ml of basic ES medium. After 4 days each 15 cm Petri dish is split at a ratio of 1 to 4. The first day after splitting, the medium is discarded. To 1 liter of conditioned basic ES medium (from the mixture of the 4 collection days), 80 ml fetal bovine serum, 17 ml non-essential amino acids, 20 ml glutamine, 6.3 μl β-mercaptoethanol, 1.25 ml insulin and 80 ml basal medium is added and the pH is adjusted to 7.4. This conditioned medium contains unmeasurable level (less than 20 pg/ml) of Rab-LIF as determined with the ELISA for human LIF of R&D Systems (Minneapolis, Minn., USA).

To this composition purified recombinant Leukemia Inhibitory Factor (LIF) can optionally be added, preferably rabbit LIF (Rab-LIF) disclosed in the patent application (WO0200847), or alternatively commercially available LIF. Antibiotics, such as penicillin/streptomycin, and insulin, may also be included in the composition.

B) ES Cell Culture Medium Preconditioned with Rab9#19

Basic medium, conditioned by the Rab9#19 fibroblast cells, is collected for 4 consecutive days as described for Rab9 described above. To 1 liter of conditioned basic ES medium (from the mixture of the 4 collection days), 80 ml fetal bovine serum, 17 ml non-essential amino acids, 20 ml glutamine, 6.3 μl β-mercaptoethanol, 1.25 ml insulin and 80 ml basal medium is added and the pH is adjusted to 7.4. Rab9#19 are Rab9 fibroblast cells which have been stably transfected with the rabbit Leukemia Inhibitory Factor gene and which secrete up to 30 ng/ml/day of Rab-LIF in the medium as determined with the ELISA for human LIF of R&D Systems (Minneapolis, Minn., USA). The production of this conditioned medium is described in detail in patent application (WO0200847).

The rabbit fibroblast cell line expressing rabbit LIF (Rab9#19 clone) was deposited with accession number LMBP 5479CB) on Apr. 07, 2000 by Thromb-X (Leopoldstraat 21, 3000 Leuven, Belgium) in the Belgian Coordinated Collections of Microorganisms (BCCM) Laboratorium voor Moleculaire Biologie-Plasmidencollectie (LMBP) Universiteit Gent, K.L.Ledeganckstraat 35, 9000 Gent, Belgium.

### Example 2

### Derivation and Culture of Mus musculus×Mus spretus Hybrid Embryonic Stem (ES) Cells

1. Mouse Strains and ES Cells

ES cells were derived from the mating of the following commercially available mouse strains: C57BL6/J (The Jackson Laboratory, Bar Harbor, Me., USA) and SPRET/Ei (Spain) The Jackson Laboratory, Bar Harbor, Me., USA).

2. Derivation of Murine ES Cells

Blastocysts were obtained from the natural matings of C57BL/6J female mice with Spretus:Ei (Spain) male mice. The blastocysts were cultured with basic medium conditioned on the Rab9#19 fibroblast cell line.

ES cells are derived from 3.5-4.5 days old blastocyst stage Mus musculus×Mus spretus hybrid embryos, which can be collected and plated individually on a 96 well dish covered with a mitotically arrested mouse embryonic fibroblast feeder monolayer. The blastocysts are allowed to attach to the monolayer, and refed every day with conditioned ES cell medium (Cfr. Bird T2267 PCT) or with ES cell medium conditioned with the Rab9#19 cell line which secreted endogenous Rab-LIF (patent application WO0200847).

After 5-6 days in culture, the inner cell mass (ICM) outgrowth is selectively removed from the (remaining) trophectoderm and replated after trypsinization with trypsin-EDTA on a 96 well dish with mitomycin arrested murine fibroblasts. Subsequently the ES cells are gradually plated on larger culture dishes. ES cells can remain undifferentiated for more than 20 passages by using conditioned ES cell medium.

The undifferentiated character of the established ES cell lines is determined by immunochemical staining for the presence of alkaline phosphatase (Vector Laboratories Inc., Burlingame, Calif.), or for the absence of vimentin and cytokeratin (both Dako A/S, Denmark). Only ES cell lines which consist for more then 90% of undifferentiated cells are maintained in culture.

Fibroblast feeder layers can be obtained from murine embryos of 12.5 days post-coitus pregnant mice. The mice are sacrificed, and the uteri collected and placed in a petri dish containing phosphate buffered saline (PBS). The embryos are dissected out of the uterus and all membranes removed. The embryos are transferred into a new dish containing PBS, the head and all internal organs removed and the carcasses washed in PBS to remove blood. The carcasses are then minced using 2 insulin syringes into cubes of 2 to 3 mm in diameter, and incubated in Trypsin-EDTA/MEM solution (10/90 V/V) at 4° C. for 2 hrs. The suspension is then incubated at 37° C. for 15 min, a single cell suspension made using a 5 ml pipette, and plated at 5×10⁶ cells per 180 mm petri dish in 25 ml Feeder Medium.

Feeder Medium consisted of 500 ml Dulbecco's Minimal Essential Medium (DMEM), 10% fetal calf serum (FCS), 13 ml penicillin/streptomycin, 13 ml glutamine, 13 ml non-essential amino acids, 2.3 μl β-mercaptoethanol. The medium is changed after 24 hr to remove debris. After 2 to 3 days of culture the fibroblasts reaches a confluent monolayer. The plates are then trypsinized, replated on 2 petri dishes, and, when confluent, the cells of each plate are frozen in 2 vials, kept at −80° C. overnight and transferred to liquid nitrogen the next day.

TABLE I
Efficiency of ES cell derivation from Mus musculus domesticus
X Mus spretus (C57BL/6J X SPRET/Ei (Spain)) hybrid mice.

| | | Number of | |
|---|---|---|---|
| | | ES cell lines | |
| | Number of blastcysts | established | |
| Mouse strain | explanted | number | % |
| (C57BL/6J X SPRET/Ei | 27 | 16 | 59 |
| (Spain)) | | | |

The basic medium conditioned by the Rab9#19 fibroblast cells allows the derivation of embryonic stem cells from the C57BL/6J×SPRET/Ei (Spain) hybrid strain. After two months of culture 16 established ES cell lines are counted. This implies an overall derivation efficiency of respectively 59%.

3. Gender of the Established ES Cell Lines

The gender of the established ES cell lines was determined by PCR and confirmed by Southern blotting with a Y-specific probe.

| ES cell line No. | gender |
|---|---|
| SPR/BL6#2 | female |
| SPR/BL6#3 | male |
| SPR/BL6#4 | male |
| SPR/BL6#7 | male |
| SPR/BL6#8 | female |
| SPR/BL6#9 | male |
| SPR/BL6#10 | male |
| SPR/BL6#11 | female |
| SPR/BL6#12 | male |
| SPR/BL6#13 | female |
| SPR/BL6#15 | female |
| SPR/BL6#18 | female |
| SPR/BL6#19 | female |
| SPR/BL6#21 | male |
| SPR/BL6#24 | male |
| SPR/BL6#26 | male |

4. Culture of ES Cells

ES cells are grown to subconfluency on mouse embryonic fibroblasts mitotically arrested with mitomycin. Culture dishes are kept at 39° C. in a humidified atmosphere of 5% CO₂ in air. The ES cells are passaged every 2-3 days onto freshly prepared feeder dishes. The ES cells are fed every day with the conditioned ES cell medium.

4. Blastocyst Injection of ES Cell Clones

The ability of the ES cells to colonize the germ line of a host embryo was tested by injection of these ES cells into host blastocysts and implanting these chimeric preimplantation embryos into pseudopregnant foster recipients according to standard procedures. The resulting chimeric offspring were test bred for germ line transmission of the ES cell genome.

ES cells of hybrid mice with an agouti coat colour were injected into host blastocysts of albino Swiss Webster or C57BL/6N mice. This allows easy identification of ES cell contribution. All ES lines tested resulted in chimaeric offspring after blastocyst injection

5. Germ Line Transmission after Blastocyst Injection

Because of the known sterility of F1 male hybrids, two female (SPR/BL6#3 SPR/BL6#18) and only 1 male (SPR/BL6#2) ES cell lines were injected into the recipient blastocysts.

Although both female as well as the male ES cell line were able to generate chimeric offspring after blastocyst injection, only the female ES cell lines showed the capability to pass the ES cell genome to the next generation (Table 1). Germline transmission from the Mus spretus as well as from the C57BL/6J genome was observed.

TABLE II
Production of chimeric mice after injection of Swiss Webster or
C57BL/6N blastocysts with Mus musculus domesticus X
Mus spretus (C57BL/6J X SPRET/Ei(Spain)) ES cells, which
were derived and cultured with basic medium conditioned on
Rab9#19 fibroblasts.

| | Passage | # blasts | # pups | | |
|---|---|---|---|---|---|
| ES cell line No. | No. | injected | born | #chimeras | germline |
| SPR/BL6#3 | 6 | | 27 | 20 | M1 & F1 |
| SPR/BL6#2 | 7 | | 16 | 6 | |
| SPR/BL6#2 | 9 | | 2 | 2 | |
| SPR/BL6#3 | 8 | | 5 | 2 | |
| SPRBL6#18 | 6 | | 3 | 8 | F1 |

M: germline transmission via male chimeras
F: germline transmission via female chimeras

Alternatively medium conditioned by the Rab9 fibroblast cells could be used to derive embryonic stem cells with germline transmission capability from Mus musculus domesticus×Mus spretus (C57BL/6J×SPRET/Ei (Spain)) hybrid mice.

Mus musculus×Mus spretus (C57BL/6J×SPRET/Ei (Spain)) ES hybrid ES cells can be used to induce gene alteration by homologous or non-homologous recombination in the

Mus spretus genoom.

Mus musculus×Mus spretus (C57BL/6J×SPRET/Ei (Spain)) ES hybrid ES cells can also be used for the expression or overexpression of genes in a Mus spretus background.

### Example 3

### Use of Hybrid Cell Lines for Genetic Manipulation of Wild Mus spretus.

The availability of Mus musculus×Mus spretus hybrid ES cell lines creates a new way to break the species barrier for genetic manipulation in wild mice like Mus spretus. Desired mutations in the Mus spretus genome can now be created via homologous recombination in Mus musculus×Mus spretus hybrid ES cells with germline transmission capability. Any kind of desired mutation may be introduced into the Mus spretus genome including null or point, mutations, as well as complex chromosomal rearrangements such as large deletions, translocations, or inversions (Hasty P, et al. Nature 350: 243-246, Valancius V and Smithies O. 1991. Mol. Cell. Biol. 11: 1402-1408, Wu H, Liu X, and Jaenisch R 1994. Proc. Natl. Acad. Sci. 91: 2819-2823 and Ramirez-Solis R, et al 1995. Nature 378: 720-724).

By specifically targeting the Mus spretus allele in the hybrid Mus musculus×Mus spretus ES cells, the phenotype of the mutation is studied in Mus spretus background. The specific targeting of Mus spretus genes is possible by means of homologous recombination. This gene targeting in mice involves several important steps:
Engineering a gene-targeting construct, which specifically recombines homologously with the Mus spretus gene. For this to occur, there is preferentially several kilobases of homology between the exogenous and genomic Mus spretus DNA, and positive selectable markers (e.g., antibiotic resistance genes) are included. In addition, negative selectable markers (e.g., “toxic” genes) can be used to select against cells that have incorporated DNA by non-homologous recombination (i.e., random insertion). Even small gaps in homology due to sequence polymorphisms between mouse strains can dramatically reduce the efficiency of homologous recombination. Therefore Mus spretus DNA is be preferentially used to construct the targeting vector.
Transfection (electroporation) of Mus musculus×Mus spretus hybrid embryonic stem (ES) cells with the targeting construct.
Assessing Mus musculus×Mus spretus hybrid ES cells for homologous recombination. The high degree of polymorphism, which exists between both parental alleles, allows an easy identification of a successful homologous recombination with the Mus spretus allele.
Generating chimeric mice by blastocyst injection (described earlier).
Assessing germline transmission of the Mus spretus genome. By choosing the appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring. The high degree of polymorphism, which exists between both parental alleles, allows an easy identification of germline transmission of the Spretus genome.
Breeding the chimeric mice, which transmit the Mus spretus genome, to homozygosity, in a pure Mus spretus background. This can be done by the mating resulting female offspring each time to a male Mus spretus.

### Example 4

### Use of Hybrid Cell Lines for the Generation of Deletion Mutants

Chromosomal deletions have already been shown to be powerful tools in the genetic analysis of complex genomes, enabling the systematic identification and localization of functional units along defined chromosomal regions. In mice, deletion complexes created by whole animal irradiation experiments have enabled a systematic characterization of functional units along defined chromosomal regions. However, classical mutagenesis in mice is impractical for generating deletion sets on a genome-wide scale.

You et al. (You Y, et al. Methods 1997; 13(4): 409-21. And You Y, et al Mamm Genome 1998; 9(3): 232-4.) have already shown that F1 hybrid ES cells of the BALB/cTa×129/SvJae genotype and of the C57BL/6J×129/SvJae genotype retain germline colonizing ability after exposure to levels of irradiation that induce chromosomal deletions. Not only are these deletions useful for the identification of genetic functions, but they also serve as mapping reagents for existing mutations or traits.

The very high polymorphism between Mus musculus and Mus spretus makes ES cells of the Mus musculus×Mus spretus genotype ideal cell lines for the generation of radiation-induced chromosomal deletions. Between different strains of Mus musculus no more than 40% of the microsatellites are polymorphic in length. Between C57BL/6 and Mus spretus up to 90% of microsatellite length can be polymorphic in length. The availability of Mus musculus×Mus spretus hybrid ES cell lines therefore will definitely contribute to a more efficient high-throughput analysis of gene function and identification of quantitative trait loci.

1-20. (canceled)

21. An isolated embryonic stem (ES) cell, which is an interspecies Mus musculus×Mus spretus hybrid ES cell.

22. The isolated ES cell according to claim 21, characterised in that said interspecies Mus musculus×Mus spretus hybrid ES cell has germ line transmission capability.

23. The isolated ES cell according to claim 21, wherein more than 40% of the microsatellites of the DNA of said ES cell are polymorphic in length.

24. The isolated ES cell according to claim 21, wherein more than 70% of the microsatellites of the DNA of said ES cell are polymorphic in length.

25. The isolated ES cell according to claim 21, wherein more than 90% of the microsatellites of the DNA of said ES cell are polymorphic in length.

26. The isolated ES cell according to claim 21, wherein the genomic background of Mus spretus is a from SPRET/Ei (Spain) Ei Mus spretus strain mice.

27. The isolated ES cell according to claim 21, wherein the genomic background of Mus musculus is from C57BL/6J.

28. The isolated ES cell according to claim 21, wherein said interspecies hybrid ES cell are derived from SPRET/Ei Mus spretus strain×C57BL6/J Mus musculus strain.

29. A population of the isolated embryonic stem (ES) cell according to claim 21.

30. A pure population of the isolated embryonic stem (ES) cell according to claim 21.

31. A method for introducing mutations into the Mus spretus genome, said method comprising the steps of:
a) transfecting an isolated embryonic stem (ES) cell of claim 22, with a gene-targeting construct, which specifically recombines homologously with said gene in Mus spretus,
b) assessing said transfected Mus musculus×Mus spretus hybrid ES cells for homologous recombination,
c) generating chimeric mice comprising said homologous recombination by blastocyst injection,
d) assessing germline transmission of the Mus spretus genome and
e) breeding said chimeric mice, which transmit the Mus spretus genome, to homozygosity, in a pure Mus spretus background.

32. The method according to claim 31, wherein the mutations are specifically introduced into the Mus spretus allele.

33. The method of claim 31, wherein the mutations are selected of the group consisting of null mutations, point mutations, translocations, inversions and deletions.

34. A method for analysing gene function or identification of quantitative trait loci comprising the step of generating radiation-induced chromosomal deletions in the interspecies Mus musculus×Mus spretus hybrid ES cells of claim 22.

35. A high-throughput analysis system for analysing gene function or identification of quantitative trait loci comprising the isolated interspecies Mus musculus×Mus spretus hybrid embryonic stem (ES) cell of claim 21, or a population of said stem cell.
